SEQUENCE LISTING (62 sequences)

SEQ ID NO: 1 - DNA, 25 bp, synthetic construct
  source 1..25: mol_type = other DNA
tgcaataccgcattcatcacctccg

SEQ ID NO: 2 - DNA, 25 bp, synthetic construct
  source 1..25: mol_type = other DNA
ttatggcggcgattatcgtcatggc

SEQ ID NO: 3 - DNA, 25 bp, synthetic construct
  source 1..25: mol_type = other DNA
gtgcttgcagcacatgttgcggatc

SEQ ID NO: 4 - DNA, 25 bp, synthetic construct
  source 1..25: mol_type = other DNA
tttcctgttattcggtaatggcgat

SEQ ID NO: 5 - DNA, 25 bp, synthetic construct
  source 1..25: mol_type = other DNA
gttgtcacaatctaaatttgccgat

SEQ ID NO: 6 - DNA, 25 bp, synthetic construct
  source 1..25: mol_type = other DNA
cagttaacgaagccgattgatcatt

SEQ ID NO: 7 - DNA, 25 bp, synthetic construct
  source 1..25: mol_type = other DNA
ggcggtttcctgttattcggtaatg

SEQ ID NO: 8 - DNA, 25 bp, synthetic construct
  source 1..25: mol_type = other DNA
cgcgatatcggtctcgacggcgtcg

SEQ ID NO: 9 - DNA, 25 bp, synthetic construct
  source 1..25: mol_type = other DNA
gccgatgatttatttgataaattag

SEQ ID NO: 10 - DNA, 25 bp, synthetic construct
  source 1..25: mol_type = other DNA
atgcggtgatacaacgcttcacctt

SEQ ID NO: 11 - DNA, 25 bp, synthetic construct
  source 1..25: mol_type = other DNA
gaatgagggcggtttgtcacaggtt

SEQ ID NO: 12 - DNA, 25 bp, synthetic construct
  source 1..25: mol_type = other DNA
tatcccagacagtatcgaatttatc

SEQ ID NO: 13 - DNA, 25 bp, synthetic construct
  source 1..25: mol_type = other DNA
tactggccgggatggggctgaccat

SEQ ID NO: 14 - DNA, 25 bp, synthetic construct
  source 1..25: mol_type = other DNA
gcctaccagtaatggcttgatatgg

SEQ ID NO: 15 - DNA, 25 bp, synthetic construct
  source 1..25: mol_type = other DNA
gcgattgtgggtgacgcggtcaatt

SEQ ID NO: 16 - DNA, 25 bp, synthetic construct
  source 1..25: mol_type = other DNA
taccgataaacttttacgatttgag

SEQ ID NO: 17 - DNA, 25 bp, synthetic construct
  source 1..25: mol_type = other DNA
gacattactcccgacgataatttat

SEQ ID NO: 18 - DNA, 25 bp, synthetic construct
  source 1..25: mol_type = other DNA
tccagccatgttttataccatgtta

SEQ ID NO: 19 - DNA, 25 bp, synthetic construct
  source 1..25: mol_type = other DNA
taccgtctgcgaatggggcaacgtc

SEQ ID NO: 20 - DNA, 25 bp, synthetic construct
  source 1..25: mol_type = other DNA
gtcatttatatcaactcatcacgcg

SEQ ID NO: 21 - DNA, 25 bp, synthetic construct
  source 1..25: mol_type = other DNA
ccaatttctttgttgcagaaaaagt

SEQ ID NO: 22 - DNA, 25 bp, synthetic construct
  source 1..25: mol_type = other DNA
cgcgattgtgggtgacgcggtcaat

SEQ ID NO: 23 - DNA, 25 bp, synthetic construct
  source 1..25: mol_type = other DNA
ttatggcggcgattatcgtcatggc

SEQ ID NO: 24 - DNA, 25 bp, synthetic construct
  source 1..25: mol_type = other DNA
taagaatgaagagaaatcactaaac

SEQ ID NO: 25 - DNA, 25 bp, synthetic construct
  source 1..25: mol_type = other DNA
atggttatttcttgatgaaccaact

SEQ ID NO: 26 - DNA, 25 bp, synthetic construct
  source 1..25: mol_type = other DNA
cacggtgcttgtcgatctgaacgat

SEQ ID NO: 27 - DNA, 25 bp, synthetic construct
  source 1..25: mol_type = other DNA
gcagccgtcgcggggatgtctggtg

SEQ ID NO: 28 - DNA, 25 bp, synthetic construct
  source 1..25: mol_type = other DNA
gaatgccccgacattactcccgacg

SEQ ID NO: 29 - DNA, 25 bp, synthetic construct
  source 1..25: mol_type = other DNA
gcatggatgtggaactggcgatatt

SEQ ID NO: 30 - DNA, 25 bp, synthetic construct
  source 1..25: mol_type = other DNA
tttatcggtaagtttgtaaatcagg

SEQ ID NO: 31 - DNA, 25 bp, synthetic construct
  source 1..25: mol_type = other DNA
catgacatgttcgtaaatgatgatt

SEQ ID NO: 32 - DNA, 25 bp, synthetic construct
  source 1..25: mol_type = other DNA
tctgaaaaaccaaattgtacagacg

SEQ ID NO: 33 - DNA, 25 bp, synthetic construct
  source 1..25: mol_type = other DNA
caaatgtataaataataaatgctat

SEQ ID NO: 34 - DNA, 25 bp, synthetic construct
  source 1..25: mol_type = other DNA
ttagataattatttagtattagcat

SEQ ID NO: 35 - DNA, 25 bp, synthetic construct
  source 1..25: mol_type = other DNA
ttagaatcttttgcctttaccgcat

SEQ ID NO: 36 - DNA, 25 bp, synthetic construct
  source 1..25: mol_type = other DNA
tacaaaattaaaattaaatggatat

SEQ ID NO: 37 - DNA, 25 bp, synthetic construct
  source 1..25: mol_type = other DNA
cttaagcataaaaatttatatgaat

SEQ ID NO: 38 - DNA, 25 bp, synthetic construct
  source 1..25: mol_type = other DNA
tcaattttatagtctgtagtctttg

SEQ ID NO: 39 - DNA, 25 bp, synthetic construct
  source 1..25: mol_type = other DNA
tgatcagccattgacttaatcggtg

SEQ ID NO: 40 - DNA, 25 bp, synthetic construct
  source 1..25: mol_type = other DNA
catttaactgattagtatctaattt

SEQ ID NO: 41 - DNA, 25 bp, synthetic construct
  source 1..25: mol_type = other DNA
attgggacaatattattaaaagcat

SEQ ID NO: 42 - DNA, 25 bp, synthetic construct
  source 1..25: mol_type = other DNA
cattaaaaaagattcataaaggaat

SEQ ID NO: 43 - DNA, 25 bp, synthetic construct
  source 1..25: mol_type = other DNA
ttcaattgttctggtttaggattgc

SEQ ID NO: 44 - DNA, 25 bp, synthetic construct
  source 1..25: mol_type = other DNA
tatagtctgtagtctttgtcgagtt

SEQ ID NO: 45 - DNA, 25 bp, synthetic construct
  source 1..25: mol_type = other DNA
aataacaattcaacacgtaattttt

SEQ ID NO: 46 - DNA, 25 bp, synthetic construct
  source 1..25: mol_type = other DNA
ttcgtggtattcgtttaggcgaagg

SEQ ID NO: 47 - DNA, 25 bp, synthetic construct
  source 1..25: mol_type = other DNA
taacaggcaatatcgccgtgcggat

SEQ ID NO: 48 - DNA, 25 bp, synthetic construct
  source 1..25: mol_type = other DNA
gttatcgcgatgaatataaactggc

SEQ ID NO: 49 - DNA, 25 bp, synthetic construct
  source 1..25: mol_type = other DNA
cagcatggcccatcctactgaaact

SEQ ID NO: 50 - DNA, 25 bp, synthetic construct
  source 1..25: mol_type = other DNA
taacaggcgatatcgccgtgcggat

SEQ ID NO: 51 - DNA, 40 bp, synthetic construct
  source 1..40: mol_type = other DNA
ttctgtccgtgcgatgggctgcaacaccaccggtgttctc

SEQ ID NO: 52 - DNA, 21 bp, synthetic construct
  source 1..21: mol_type = other DNA
acgcgacttggttgggtattc

SEQ ID NO: 53 - DNA, 40 bp, synthetic construct
  source 1..40: mol_type = other DNA
ttctgtccgtgcgatgggctgcaacaccaccggtgttctt

SEQ ID NO: 54 - DNA, 21 bp, synthetic construct
  source 1..21: mol_type = other DNA
acgcgacttggttgggtattc

SEQ ID NO: 55 - DNA, 20 bp, synthetic construct
  source 1..20: mol_type = other DNA
cgggattcggtgtaacgcat

SEQ ID NO: 56 - DNA, 32 bp, synthetic construct
  source 1..32: mol_type = other DNA
gtactgggtaaataccatccgcacggcgatct

SEQ ID NO: 57 - DNA, 20 bp, synthetic construct
  source 1..20: mol_type = other DNA
cgggattcggtgtaacgcat

SEQ ID NO: 58 - DNA, 32 bp, synthetic construct
  source 1..32: mol_type = other DNA
gtactgggtaaataccatccgcacggcgatcg

SEQ ID NO: 59 - DNA, 24 bp, synthetic construct
  source 1..24: mol_type = other DNA
gacgctggaacagcttgaagctct

SEQ ID NO: 60 - DNA, 30 bp, synthetic construct
  source 1..30: mol_type = other DNA
gcccagagccagtttatattcatcgcgata

SEQ ID NO: 61 - DNA, 20 bp, synthetic construct
  source 1..20: mol_type = other DNA
cagcatggcccatcctactg

SEQ ID NO: 62 - DNA, 17 bp, synthetic construct
  source 1..17: mol_type = other DNA
tgccgccatcctgatcc

(19) United States
(12) Patent Application Publication
(10) Pub. No.: US 2026/0290509 A1
(43) Pub. Date: Sep. 24, 2026

(54) IDENTIFICATION OF MARKER SEQUENCES FOR RAPID CLASSIFICATION OF MICROBIAL PATHOGENS THROUGH MACHINE LEARNING ANALYSIS OF GENOME ASSEMBLIES

(71) Applicant: The Regents of the University of California, Oakland, CA (US)

(72) Inventors: Jason C. Hyun, Rockville, MD (US); Bernhard Orn Palsson, San Diego, CA (US)

(21) Appl. No.: 19/477,448

(22) PCT Filed: May 31, 2024
(86) PCT No.: PCT/US2024/032104

§ 371 Date: Oct. 21, 2025

Related U.S. Application Data
(60) Provisional application No. 63/470,417, filed on Jun. 1, 2023.

(51) Int. Cl. G16B 40/20; G16B 20/20; G16B 30/00

(52) U.S. Cl. CPC G16B 40/20; G16B 20/20; G16B 30/00

(57) ABSTRACT

The disclosure provides methods to identify marker sequences for rapid classification of microbial strains through machine learning analysis of genome assemblies.

### CROSS REFERENCE TO RELATED APPLICATIONS

This application claims priority under 35 U.S.C. § 119 from Provisional Application Ser. No. 63/470,417, filed Jun. 1, 2023 the disclosures of which are incorporated herein by reference.

### STATEMENT OF GOVERNMENT SUPPORT

This invention was made with Government support under Grant Nos U01-AI124316, awarded by the National Institute of Allergy and Infectious Diseases. The Government has certain rights in the invention.

### TECHNICAL FIELD

The disclosure provides methods to identify marker sequences for rapid classification of microbial strains through machine learning analysis of genome assemblies.

### INCORPORATION BY REFERENCE OF SEQUENCE LISTING

Accompanying this filing is a Sequence Listing entitled, “00015-421WO1.xml” created on May 31, 2024 and having 56,079 bytes of data, machine formatted on IBM-PC, MS-Windows operating system. The sequence listing is hereby incorporated by reference in its entirety for all purposes.

### BACKGROUND

A challenge in dealing with acute bacterial infections in a clinical setting is to obtain rapid identification of the invading pathogen. Once the taxonomic identity of the pathogen has been determined, a second challenge is to determine if it has known antimicrobial resistance (AMR) characteristics, which are important for determining the appropriate treatment modality. Currently, these determinations are made by time consuming laboratory procedures that are typically performed in a pathology laboratory that require a day or two for analysis.

### SUMMARY

The disclosure provides methods to identify marker sequences for rapid classification of microbial strains through machine learning analysis of genome assemblies. In a particular embodiment, a method disclosed herein provides for generating a pangenome compendium from genome assemblies of all publicly available microbial strains of interest. This pangenome compendium of strains is then run through a machine-learning pipeline alongside a classification schema (e.g., pathogenic vs. nonpathogenic strains) to identify genes which can robustly classify microbial strains into the desired schema. These genes are then used to develop primer sequences which can rapidly identify these microbial strains through PCR tests performed in a laboratory.

For example, given a prokaryotic pangenome of interest (e.g., a known pathogenic species like E. coli or S. aureus) and a desired classification scheme (e.g., pathotypes, anti-microbial resistance (AMR), etc.), the methods disclosed herein can identify a limited number of short DNA sequences that can accurately reproduce the classification scheme. As an exemplary embodiment, the disclosure presented herein reproduce three different classification schemes: (1) determining the phylogenetic group of Escherichia coli traditionally defined by Clermont Typing, (2) determining the clonal complex of Staphylococcus aureus, and (3) determining resistance against ciprofloxacin for Escherichia coli isolates in the B2 phylogroup.

The disclosure provides a method to identify a set of oligonucleotide marker sequences for rapid classification of microbial pathogens from a dataset of microbial genome assemblies, comprising carrying out steps (1)-(8): (1) clustering open reading frames by protein sequence to form a pangenome from the dataset of microbial genome assemblies, wherein individual clusters of open reading frames are designated as “genes”; (2) filtering the genes to identify sets of genes that are associated by a targeted classification scheme; (3) training machine learning models to iteratively identify minimal sets of related “genes” that are (a) able to accurately reproduce a targeted classification scheme, and (b) can be accurately identified by a marker sequence, given all observed variants of the gene and against the background of the pangenome, wherein the identified minimal sets of associated genes that meet criteria (a) and (b) are designated as candidate genes; (4) identifying sequence variants from each of the candidate genes, and from which, subsequences having a defined number of nucleotides are enumerated, wherein the enumerated subsequences of length k are designated “kmers”; (5) identifying conserved kmers for all candidate genes across the pangenome; (6) filtering the conserved kmers to identify those conserved kmers that accurately reproduce the presence/absence of their corresponding gene, wherein these identified conserved kmers are designated as accurate kmers; (7) training machine learning models to classify genomes from the accurate kmers and filtering out genes that cannot be reproduced accurately from a single accurate kmers, wherein steps (3)-(6) are repeated until a kmers-based machine learning model able to reproduce the target classification scheme with accuracy greater than 95%, 98%, or 99% is generated; and (8) selecting a minimal set of oligonucleotide marker sequences from the accurate machine learning model, wherein the minimal set of oligonucleotide marker sequences can be used to generate primers for the classification of microbial pathogens. In one embodiment, the microbial pathogens are selected from bacterial pathogens, viral pathogens, and fungi pathogens. In another or further embodiment, the dataset comprises microbial genome assemblies from bacteria, viruses or fungi. In another embodiment, for step (1), the microbial genomes assemblies contain genomes that have been filtered for quality based on genome status, size, number of contigs, number of genes and consistency. In still another or further embodiment, in step (1), the microbial genomes assemblies have been annotated to mark open reading frames in the genomes. In still another or further embodiment, in step (1), the open reading frames are clustered using a program that clusters and compares protein or nucleotide sequences. In yet another or further embodiment, in step (2), the targeted classification scheme comprises genes that are related to drug-resistance, virulence, pathotype, or phylogenetically-defined subgroups. In another or further embodiment, in step (2), the genes are filtered for those associated with the classification scheme, wherein association is based upon a pair of classes for which the gene is observed in X % of class 1 genomes and Y % of class 2 genomes, where X>Y+10. In yet another or further embodiment, in step (3), the machine learning models are trained to identify 4-8 genes that most accurately recover the targeted classification scheme. In still another or further embodiment, in step (4), the subsequences have a number of nucleotides selected from 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, and 35 nt, or a range of nucleotides that includes or is between any two of the foregoing numbers. In yet a further embodiment, the subsequences have a number of nucleotides selected from 20 nt to 30 nt. In still another or further embodiment, in step (5), the conserved kmers occur in greater than 808, 858, 90%, 918, 92%, 93%, 94% or 95% of instances of the gene. In another or further embodiment, in step (6), the accuracy of the conserved kmers to reproduce the presence/absence of their corresponding gene is determined based upon the F1 score. In still another or further embodiment, in step (7), the machine learning models are trained to identify 4-8 kmers that most accurately recover the targeted classification scheme. In still another or further embodiment, of any of the foregoing, the method further comprises: classifying a microbial pathogen using one or more oligonucleotide marker sequences selected in step (8).

The disclosure also provides that the foregoing method can be implemented by a computer. In one embodiment, the computer system implementing the method can be linked to an oligonucleotide synthesizer.

The disclosure also provides a computer readable medium comprising instructions to cause a computer to identify a set of oligonucleotide marker sequences for rapid classification of microbial pathogens from a dataset of microbial genome assemblies, the computer instructions causing the computer to: (1) cluster open reading frames by protein sequence to form a pangenome from the dataset of microbial genome assemblies, wherein individual clusters of open reading frames are designated as “genes”; (2) filter the genes to identify sets of genes that are associated by a targeted classification scheme; (3) train machine learning model(s) to iteratively identify minimal sets of related “genes” that are (a) able to accurately reproduce a targeted classification scheme, and (b) can be accurately identified by a marker sequence, given all observed variants of the gene and against the background of the pangenome, wherein the identified minimal sets of associated genes that meet criteria (a) and (b) are designated as candidate genes; (4) identify sequence variants from each of the candidate genes, and from which, subsequences having a defined number of nucleotides are enumerated, wherein the enumerated subsequences of length k are designated “kmers”; (5) identify conserved kmers for all candidate genes across the pangenome; (6) filter the conserved kmers to identify those conserved kmers that accurately reproduce the presence/absence of their corresponding gene, wherein these identified conserved kmers are designated as accurate kmers; (7) train machine learning models to classify genomes from the accurate kmers and filtering out genes that cannot be reproduced accurately from a single accurate kmers, wherein steps (3)-(6) are repeated until a kmers-based machine learning model able to reproduce the target classification scheme with accuracy greater than 95%, 98%, or 99% is generated; and (8) select a minimal set of oligonucleotide marker sequences from the accurate machine learning model, wherein the minimal set of oligonucleotide marker sequences can be used to generate primers for the classification of microbial pathogens.

The disclosure also provides a catalog of oligonucleotide marker sequences obtained by the method or a computer running the computer readable medium of the disclosure.

The disclosure also provides a method of identifying a pathogen in a sample, the method comprising comparing sequence reads obtained from the genome of the pathogen in the sample to the catalog of oligonucleotide markers in the catalog to identify the pathogen with a complementary sequence.

### DESCRIPTION OF DRAWINGS

FIG. 1A-B presents exemplary flowcharts/workflows for identifying a minimal set of marker sequences or “primers” that accurately reproduces a known genome classification scheme using massive public datasets and genetic algorithms. (A) A flowchart demonstrating how a minimal set of marker sequences can be generated from a collection of genome assemblies, with various decisions indicated to improve machine learning model accuracy. (B) Additional workflows to identify a minimal set of marker sequences or “primers” using different intermediate data types.

FIG. 2 displays confusion matrices for classifying E. coli into phylogroups from primer sets of 4, 5, and 6 sequences. For marker sets of size 4 and 5, predictions corresponding to any of the cryptic clades, non-Escherichia “Non-Esc.”, or unknown were not generated.

FIG. 3 displays confusion matrices for classifying E. coli into phylogroups from primer sets of 7 or 8 sequences. The label “Non-Esc.” corresponds to a non-Escherichia classification.

FIG. 4 presents interpretation of the 4-sequence primer set for predicting E. coli phylogroup. White indicates the primer sequence is present, black indicates absence. Combinations not shown were not encountered in the training data.

FIG. 5 presents interpretation of the 5-sequence primer set for predicting E. coli phylogroup. White indicates the primer sequence is present, black indicates absence. Combinations not shown were not encountered in the training data.

FIG. 6 presents interpretation of the 6-sequence primer set for predicting E. coli phylogroup. White indicates the primer sequence is present, black indicates absence. Combinations not shown were not encountered in the training data.

FIG. 7 presents interpretation of the 7-sequence primer set for predicting E. coli phylogroup. White indicates the primer sequence is present, black indicates absence. Combinations not shown were not encountered in the training data.

FIG. 8 presents interpretation of the 8-sequence primer set for predicting E. coli phylogroup. White indicates the primer sequence is present, black indicates absence. Combinations not shown were not encountered in the training data.

FIG. 9 displays confusion matrices for classifying S. aureus into clonal complexes from primer sets of 4, 5, and 6 sequences.

FIG. 10 presents interpretation of the 4-sequence primer set for predicting S. aureus clonal complex. White indicates the primer sequence is present, black indicates absence. Combinations not shown were not encountered in the training data.

FIG. 11 presents interpretation of the 5-sequence primer set for predicting S. aureus clonal complex. White indicates the primer sequence is present, black indicates absence. Combinations not shown were not encountered in the training data.

FIG. 12 provides interpretation of the 6-sequence primer set for predicting S. aureus clonal complex. White indicates the primer sequence is present, black indicates absence. Combinations not shown were not encountered in the training data.

FIG. 13A-B provides performance and interpretation of four primers for predicting the ciprofloxacin resistance phenotype of E. coli B2 strains. (A) Confusion matrix for the binary classification of E. coli B2 strains by ciprofloxacin resistance phenotype from the four primers. (B) Interpretation of the four primers to predict ciprofloxacin resistance phenotype. White indicates the primer sequence is present, black indicates absence. Combinations not shown were not encountered in the training data.

FIG. 14 provide genetic and antimicrobial resistance profiles of 442 candidate strains for ciprofloxacin resistance marker sequence validation. Genetic cluster membership and predicted ciprofloxacin resistance phenotypes are shown for all 442 candidates (top) and the 72 selected validation strains (bottom).

FIG. 15 provides a comparison of predicted vs. experimentally observed ciprofloxacin resistance phenotypes for 72 validation strains. Prediction method “ML” refers to the pangenome-based XGBoost model, “Marker” refers to the original proposed interpretation of the four marker sequences, and “Marker+1” refers to the modified interpretation adding a fifth marker to also capture the S80I double SNP mutation. Strains have been sorted by observed resistance phenotype first, then by strain genetic cluster.

FIG. 16 shows detection of ciprofloxacin resistance marker sequence targets using a PCR-based approach.

FIG. 17 illustrates a block diagram of an example machine upon which one or more embodiments (e.g., discussed methodologies) can be implemented.

FIG. 18 depicts a block diagram for a system or related method of an embodiment of the present invention in whole or in part.

### DETAILED DESCRIPTION

As used herein and in the appended claims, the singular forms “a,” “an,” and “the” include plural referents unless the context clearly dictates otherwise. Thus, for example, reference to “a gene” includes a plurality of such genes and reference to “the gene variant” includes reference to one or more gene variants and equivalents thereof known to those skilled in the art, and so forth.

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated by reference in full for the purpose of describing and disclosing methodologies that might be used in connection with the description herein. The publications are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is like, or identical with, a term that has been expressly defined in this disclosure, the definition of the term as expressly provided in this disclosure will control in all respects.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless otherwise defined, all technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, NY 2006); Smith, March's Advanced Organic Chemistry Reactions, Mechanisms and Structure 7^{th} ed., J. Wiley & Sons (New York, NY 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, NY 2012), provide one skilled in the art with a general guide to many of the terms used in the present application.

All headings and subheading provided herein are solely for ease of reading and should not be construed to limit the invention. Although methods and materials similar or equivalent to those described herein can be used in the practice or testing of the invention, suitable methods and materials are described below.

It should be understood that this disclosure is not limited to the particular methodology, protocols, and reagents, etc., described herein and as such may vary. The terminology used herein is for the purpose of describing particular embodiments or aspects only and is not intended to limit the scope of the present disclosure.

Other than in the operating examples, or where otherwise indicated, all numbers expressing quantities of ingredients or reaction conditions used herein should be understood as modified in all instances by the term “about.” The term “about” when used to describe embodiments of the disclosure, in connection with percentages means ±1%. The term “about,” as used herein can mean within an acceptable error range for the particular value as determined by one of ordinary skill in the art, which can depend in part on how the value is measured or determined, e.g., the limitations of the measurement system. Alternatively, “about” can mean a range of plus or minus 208, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” means within an acceptable error range for the particular value. Also, where ranges and/or subranges of values are provided, the ranges and/or subranges can include the endpoints of the ranges and/or subranges.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “amplifying” refers to the process of synthesizing nucleic acid molecules that are complementary to one (or both strands) of a template nucleic acid molecule. Amplifying a nucleic acid molecule typically includes denaturing the template nucleic acid, particularly if the template nucleic acid is double-stranded, annealing one or more primers (e.g., primers generated by the methods of the disclosure) to the template nucleic acid at a temperature that is below the melting temperatures of the primers, and enzymatically elongating from the primers to generate an amplification product. Generally, synthesis initiates at the 3′ end of a primer and proceeds in a 5′ to 3′ direction along the template nucleic acid strand. Amplification typically requires the presence of deoxyribonucleoside triphosphates, a polymerase enzyme (e.g., DNA or RNA polymerase or T7 for in vitro transcription in TMA) and an appropriate buffer and/or co-factors for optimal activity of the polymerase enzyme (e.g., MgCl₂ and/or KCl).

With respect to the term “different taxon of pathogens”, the term is distinct from the “particular taxon of pathogens”. Here, the different taxon of pathogenic microorganisms does not overlap with the particular taxon of pathogens. For example, if a particular taxon of pathogenic microorganisms includes the family of Flavivirus, the different taxon of pathogenic microorganisms does not include Flavivirus but can include another family of viruses, such as Alphaviruses, bacterial, fungal, archaea, algal, protozoan, and/or parasitic pathogens. If the particular taxon of pathogenic microorganisms and different taxon of pathogenic microorganisms are from the same domain (e.g., bacterial domain), the two taxa identified by the method are distinct.

The term “microorganism” or “microbial organism” is used in its broadest sense and includes Gram negative aerobic bacteria, Gram positive aerobic bacteria, Gram negative microaerophillic bacteria, Gram positive microaerophillic bacteria, Gram negative facultative anaerobic bacteria, Gram positive facultative anaerobic bacteria, Gram negative anaerobic bacteria, Gram positive anaerobic bacteria, Gram positive asporogenic bacteria, Actinomycetes, fungal microorganism, protazoan microorganism and the like.

As used herein, the term “pathogen” refers to a virus, bacterium, protozoa, prion, archaea, fungus, algae, parasite, or other microbe (helminth) that causes or induces disease or illness in a subject or that may be found in biological and/or environmental samples. The term includes both the disease-causing organism per se and toxins produced by the pathogen (e.g., Shiga toxins) present in a sample. Detection of a pathogen as set forth in the methods disclosed herein includes detection of a portion of the genome of the pathogen or a nucleic acid molecule that is complementary or substantially complementary (i.e., at least 90% complementary) to a portion of the genome of the pathogen.

The terms “polynucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA (TRNA), ribosomal RNA (rRNA), short interfering RNA (siRNA), short-hairpin RNA (shRNA), micro-RNA (miRNA), ribozymes, CDNA, recombinant polynucleotides, branched polynucleotides, plasmids, vectors, isolated DNA of any sequence, isolated RNA of any sequence, nucleic acid probes, and primers. A polynucleotide may comprise methylated nucleotides and nucleotide analogs.

The terms “polypeptide”, “peptide” and “protein” are used interchangeably herein to refer to polymers of amino acids of any length. The polymer may be linear or branched. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “primer” refers to oligomeric compounds, primarily to oligonucleotides containing naturally occurring nucleotides such as adenine, guanine, cytosine, thymine and/or uracil, but may also include modified oligonucleotides (e.g., modified nucleotides, nucleosides, synthetic nucleotides having modified base moieties and/or modified sugar moieties (See, Protocols for Oligonucleotide Conjugates, Methods in Molecular Biology, Vol 26, (Sudhir Agrawal, Ed., Humana Press, Totowa, N. J., (1994)); and Oligonucleotides and Analogues, A Practical Approach (Fritz Eckstein, Ed., IRL Press, Oxford University Press, Oxford) that are able to prime polynucleotide (e.g., DNA) synthesis by an enzyme, typically in a template-dependent manner, i.e., the 3′ end of the primer provides a free 3′-OH group to which further nucleotides are attached by the enzyme (e.g., DNA polymerase or reverse transcriptase) establishing a 3′ to 5′ phosphodiester linkage whereby nucleoside triphosphates are used and pyrophosphate is released. Oligonucleotides can be prepared by any suitable method, including, for example, cloning and restriction of appropriate sequences and direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066. A review of synthesis methods is provided in Goodchild, 1990, Bioconjugate Chemistry 1(3):165-187.

A primer is typically a single-stranded deoxyribonucleic acid. The appropriate length of a primer depends on the intended use of the primer but typically ranges from 6 to 50 nucleotides. Short primer molecules (e.g., having a length within a range of 11-17 nucleotides) generally require cooler temperatures to form sufficiently stable hybrid complexes with a template (or target) nucleic acid.

The terms “subject,” “individual,” and “patient” are used interchangeably herein to refer to a vertebrate, a mammal, more preferably a human. Mammals include, but are not limited to, murines, simians, humans, farm animals, sport animals, and pets.

The terms “taxon” or “taxa”, “taxonomic group,” and “taxonomic unit” are used interchangeably to refer to a group of one or more organisms that comprises a node in a clustering tree. The level of a cluster is determined by its hierarchical order. In one embodiment, a taxon is a group tentatively assumed to be a valid taxon for purposes of phylogenetic analysis. In another embodiment, a taxon is any of the extant taxonomic units under study. In yet another embodiment, a taxon is given a name and a rank. For example, a taxon can represent a domain, a sub-domain, a kingdom, a sub-kingdom, a phylum, a sub-phylum, a class, a sub-class, an order, a sub-order, a family, a subfamily, a genus, a subgenus, or a species. In some embodiments, taxa can represent one or more organisms from the kingdoms eubacteria, protista, or fungi at any level of a hierarchal order.

A challenge in dealing with acute infections (e.g., bacterial infections) in a clinical setting is to obtain rapid identification of the invading pathogen. Once the taxonomic identity of the pathogen has been determined, a second challenge is to determine if it has known antimicrobial resistance (AMR) characteristics, which are important for determining the appropriate treatment modality. Currently, these determinations are made by time consuming laboratory procedures that are typically performed in a pathology laboratory that require a day or two to perform.

An alternative is to perform genetic assessment of the invading pathogens. Such tests can be performed more rapidly than pathology laboratory procedures requiring pathogen cultures and test screening, thereby enabling earlier implementation of appropriate treatment modality. Genetic tests require a priori knowledge of what DNA sequences to look for in an invading pathogen. The identity of such sequences can be obtained through the assessment of genomic sequences from a large set of strains of the invading pathogenic strain. Currently, there is a rapid increase in the number of genomic sequences of strains of major human bacterial pathogens that can be used to achieve such an assessment.

The disclosure provides methods to identify marker sequences for rapid classification of microbial strains through machine learning analysis of genome assemblies. Once the proper phylogroup (i.e., which strain subgroup of a species, which is known to impact the severity of infection) designation of the microbe has been made, a follow-up panel of genetic tests can be applied to determine the AMR status of the microbe.

The methods of the disclosure use various machine learning processes. Machine learning may include, but are not limited to, one or more of any combination of the following: Naïve Bayes Classifier, Neural Networks, Decision Trees, Generalized Linear Models, Nearest Neighbors, Support Vector Machines, or “ensemble” methods such as Random Forests that combine the predictions of multiple supervised machine learning models. Still yet, the training may be accomplished through simulation.

In addition, the methods of the disclosure can use various genetic/genome databases. The database of reference sequences can comprise any of a variety of reference sequences. In some embodiments, the reference sequences are from one or more of bacteria, archaea, chromalveolata, viruses, fungi, plants, fish, amphibians, reptiles, birds, mammals, and humans. In some cases, the database of reference sequences consists of sequences from a reference individual or a reference sample source. In this case, the method may further comprise identifying polynucleotides from the sample source as being derived from the reference individual or the reference sample source. In some embodiments, the database of reference sequences comprises one or more mutations with respect to known polynucleotide sequences, such that a plurality of variants of the known polynucleotide sequences are represented in the database of reference sequences. The database of reference sequences can comprise marker gene sequences for taxonomic classification of bacterial sequences, such as 16S rRNA sequences.

In some embodiments, the database of reference sequences consists of sequences associated with a condition. One or more such sequences may form a biosignature for a condition, a plurality of which may together form the reference database. In some cases, the record database is associated with a condition of the sample source to establish a biosignature for a condition. When sequences are associated with a condition, the method may further comprise identifying a condition of the sample source by comparison of the record database to a biosignature, including identifying the sample source as having the condition. The condition may be contamination, such as food contamination, surface contamination, or environmental contamination. In some embodiments, the condition is infection.

Where the reference database consists of sequences associated with infectious disease or contamination, the sequences may be derived from and associated with any of a variety of infectious agents. The infectious agent can be bacterial. Non-limiting examples of bacterial pathogens include Mycobacteria (e.g. M. tuberculosis, M. bovis, M. avium, M. leprae, and M. africanum), rickettsia, mycoplasma, chlamydia, and legionella. Other examples of bacterial infections include, but are not limited to, infections caused by Gram positive bacillus (e.g., Listeria, Bacillus such as Bacillus anthracis, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, and Neisseria species. Specific examples of infectious bacteria include, but are not limited to: Helicobacter pyloris, Legionella pneumophilia, M. intracellular, M. kansaii, M. gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, Erysipelothrix rhusiopathiae, Clostridium tetani, Enterobacter aerogenes, Klebsiella pneumoniae, Pasteurella multocida, Fusobacterium nucleatum, Streptobacillus moniliformis, Treponema pallidium, Treponema pertenue, Leptospira, Rickettsia, Actinomyces israelii, Acinetobacter, Bacillus, Bordetella, Borrelia, Brucella, Campylobacter, Chlamydia, Chlamydophila, Clostridium, Corynebacterium, Enterococcus, Haemophilus, Helicobacter, Mycobacterium, Mycoplasma, Stenotrophomonas, Treponema, Vibrio, Yersinia, Acinetobacter baumanii, Bordetella pertussis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Corynebacterium diphtheriae, Enterobacter sazakii, Enterobacter agglomerans, Enterobacter cloacae, Enterococcus faecalis, Enterococcus faecium, Escherichia coli, Francisella tularensis, Helicobacter pylori, Legionella pneumophila, Leptospira interrogans, Mycobacterium leprae, Mycobacterium tuberculosis, Mycobacterium ulcerans, Mycoplasma pneumoniae, Pseudomonas aeruginosa, Rickettsia rickettsii, Salmonella typhi, Salmonella typhimurium, Salmonella enterica, Shigella sonnei, Staphylococcus epidermidis, Staphylococcus saprophyticus, Stenotrophomonas maltophilia, Vibrio cholerae, Yersinia pestis, and the like.

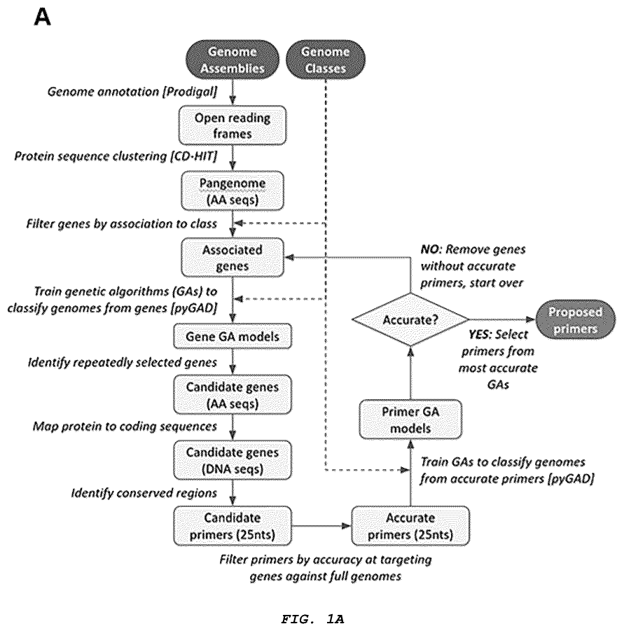

FIG. 1A-B provides exemplary workflow/flowchart to identify marker sequences for rapid classification of microbial strains through machine learning analysis of genome assemblies. As shown, for a species for which a classification scheme is to be developed, a relevant public genomic data is used, assembled and structured into a “pangenome” as described in Hyun et al. (BMC Genomics 23(1):7 (2022)), which is incorporated herein in its entirety.

In one embodiment of the disclosure, starting with all available public genome sequences for the species on the Bacterial and Viral Bioinformatics Resource Center (BV-BRC; see, e.g., internet at bv-brc.org), genomes are filtered for quality based on genome status, size, number of contigs, number of genes, and consistency. Across all remaining high-quality genomes, open reading frames that are either publicly annotated or generated using a program (e.g., Prodigal) are clustered by protein sequence using CD-HIT (Cluster Database at High Identity with Tolerance) into “genes”. The gene content across all genomes is then represented as a binary matrix of presence/absence calls between genes and genomes. All genomes are classified following a target classification scheme with an appropriate existing method (e.g., by examining existing metadata or implementing the current best practice), and genes are filtered for those associated with the classification (defined as: there exists a pair of classes for which the gene is observed in X % of class 1 genomes and Y % of class 2 genomes, where X>Y+10). Examples of a target classification scheme include, but are not limited to, classifying by pathogenic vs. nonpathogenic, classifying by drug-resistant vs. drug-susceptible, classifying by virulence, classifying by clonal complex, phylogroup, or other phylogenetically-defined subgroup, etc.

Next, a genetic algorithm (GA) approach is used to iteratively identify minimal sets of genes that are (1) able to accurately reproduce the target classification scheme, and (2) can be accurately identified by a marker sequence, given all observed variants of the gene and against the background of the species' pangenome. In one embodiment, prior to testing a non-identified sample, 5-15 (e.g., 5, 6, 7, 8, 9, 10, 11, 12, 13, 14 or 15) randomly seeded GA models are trained to identify 4-8 (e.g., 4, 5, 6, 7, or 8) genes that most accurately recover the target classification scheme. More particularly, the machine learning model can be implemented by splitting genomes into training/testing sets, with the following parameter set: num_generations=200, sol_per_pop=512, num_parents_mating=256, keep_elitism=1, parent_selection_type=‘sss’, crossover_type=‘uniform’, mutation_type=‘random’, mutation_probability=0.20. The identified gene sets across the resulting GA models are then combined as a short list of candidate genes from which marker sequences are derived. For each candidate gene, all DNA sequence variants are identified and subsequences of length 11-50 nt (e.g., about 25 nt) are enumerated, referred to as “kmers”. Conserved kmers (occurring in >90% of instances of the gene) are identified, and the presence/absence of such kmers for all candidate genes is/are computed across all genomes and are filtered for those that accurately reproduce the presence/absence of their corresponding gene (e.g., based on an F1 score). GA models are then similarly trained to identify 4-8 of the remaining kmers that accurately recover the target classification. If the final kmer-based GA models are not sufficiently accurate, genes that cannot be reproduced accurately by a single kmer are filtered out and the entire process is repeated, starting from the GA models trained on genes. If a sufficiently accurate GA model is found, then the selected kmers of that model is a novel set of marker sequences that accurately reproduces the original classification scheme. Such marker sequences can then be used to synthesize oligonucleotide probes and/or primers.

This two-phase approach of analyzing the classification at the gene-level then at the kmer-level provides two benefits towards developing marker sequence sets. First, there are far fewer genes than unique kmers observed within a pangenome, so the two problems of identifying predictive genes followed by identifying predictive kmers among those genes are both much more computationally tractable than the direct approach of enumerating all observed kmers and identifying predictive kmers directly. Second, this approach yields marker sequences that closely track specific genes, allowing for a more straightforward biological interpretation of proposed marker sequences and potentially facilitate commercial adoption.

The methods of the disclosure are innovative in that they fully utilize big data and generate large pangenome collections which fully represent all publicly available sequences of strains. This improves the accuracy of the classification schema over traditional methods and better identifies rarer classification subtypes. Moreover, the methods of the disclosure provide faster classification of pathogens isolated from a patient than is currently possible. Millions of pathogens are classified in pathology labs in the US annually. Similarly, the methods of the disclosure can rapidly identify drug-resistant strains. Other applications can also be envisaged, such as screening wastewater or other samples (e.g., patient samples, environmental samples, etc.) for specific strains of bacteria or viruses (e.g., COVID/Sars COV-2).

The markers or primers developed by the methods of the disclosure can be used to screen for a pathogen or pathogen's antimicrobial susceptibility. The methods (and resulting oligonucleotide compositions and kits) provide improved identification and/or quantification of target nucleic acid molecules in a sample from a subject, e.g., by RT-qPCR and/or next-gen-sequencing (NGS).

A number of different assay techniques can use the oligonucleotide primers obtained by the methods of the disclosure including, but not limited to, lateral flow assays, PCR, NGS, southern blots, northern blots, and the like.

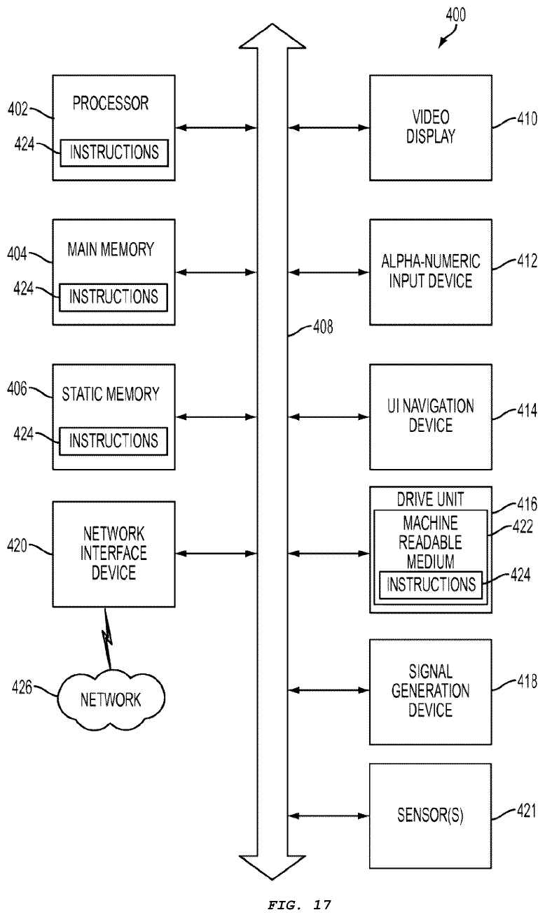

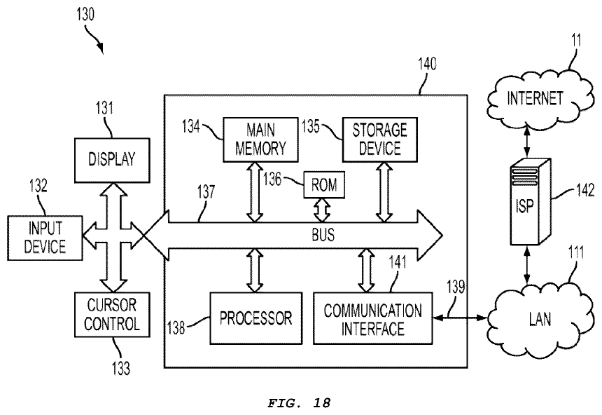

FIG. 17 illustrates a block diagram of an example machine 400 upon which one or more embodiments (e.g., the foregoing discussed methodologies) can be implemented (e.g., run). Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (Saas). For example, at least some of the operations can be performed by a group of computers (as examples of machines including processors), with these operations being accessible via a network (e.g., the Internet) and via one or more appropriate interfaces (e.g., Application Program Interfaces (APIs)).

Example embodiments (e. g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400, Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 810, input device 417 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.16 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software,

FIG. 18 is a block diagram that illustrates a system 130 including a computer system 140 and the associated Internet 11 connection upon which an embodiment may be implemented. Such configuration is typically used for computers (hosts) connected to the Internet 11 and executing a server or a client (or a combination) software. A source computer such as laptop, an ultimate destination computer and relay servers, for example, as well as any computer or processor described herein, may use the computer system configuration and the Internet connection shown in FIG. 18. The system 140 may be used as a portable electronic device such as a notebook/laptop computer, a media player (e. g., MP3 based or video player), a cellular phone, a Personal Digital Assistant (PDA), a sample device, an image processing device (e.g., a digital camera or video recorder), and/or any other handheld computing devices, or a combination of any of these devices. Note that while FIG. 17 illustrates various components of a computer system, it is not intended to represent any particular architecture or manner of interconnecting the components; as such details are not germane to the present invention. It will also be appreciated that network computers, handheld computers, cell phones and other data processing systems which have fewer components or perhaps more components may also be used. Computer system 140 includes a bus 137, an interconnect, or other communication mechanism for communicating information, and a processor 138, commonly in the form of an integrated circuit, coupled with bus 137 for processing information and for executing the computer executable instructions. Computer system 140 also includes a main memory 134, such as a Random Access Memory (RAM) or other dynamic storage device, coupled to bus 137 for storing information and instructions to be executed by processor 138.

Main memory 134 also may be used for storing temporary variables or other intermediate information during execution of instructions to be executed by processor 138. Computer system 140 further includes a Read Only Memory (ROM) 136 (or other non-volatile memory) or other static storage device coupled to bus 137 for storing static information and instructions for processor 138. A storage device 135, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, is coupled to bus 137 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. Typically computer system 140 includes an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term “processor” is meant to include any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor.

Computer system 140 may be coupled via bus 137 to a display 131, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device 132, including alphanumeric and other keys, is coupled to bus 137 for communicating information and command selections to processor 138. Another type of user input device is cursor control 133, such as a mouse, a trackball, or cursor direction keys for communicating direction information and command selections to processor 138 and for controlling cursor movement on display 131. This input device typically has two degrees of freedom in two axes, a first axis (e.g., x) and a second axis (e. g., y), that allows the device to specify positions in a plane.

The computer system 140 may be used for implementing the methods and techniques described herein. According to one embodiment, those methods and techniques are performed by computer system 140 in response to processor 138 executing one or more sequences of one or more instructions contained in main memory 134. Such instructions may be read into main memory 134 from another computer-readable medium, such as storage device 135. Execution of the sequences of instructions contained in main memory 134 causes processor 138 to perform the process steps described herein. In alternative embodiments, hard-wired circuitry may be used in place of or in combination with software instructions to implement the arrangement. Thus, embodiments of the invention are not limited to any specific combination of hardware circuitry and software.

The term “computer-readable medium” (or “machine-readable medium”) as used herein is an extensible term that refers to any medium or any memory, that participates in providing instructions to a processor, (such as processor 138) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that comprise bus 137. Transmission media can also take the form of acoustic or light waves, such as those generated during radio-wave and infrared data communications, or other form of propagated signals (e.g., carrier waves, infrared signals, digital signals, etc.). Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor 138 for execution. For example, the instructions may initially be carried on a magnetic disk of a remote computer. The remote computer can load the instructions into its dynamic memory and send the instructions over a telephone line using a modem. A modem local to computer system 140 can receive the data on the telephone line and use an infra-red transmitter to convert the data to an infra-red signal. An infra-red detector can receive the data carried in the infra-red signal and appropriate circuitry can place the data on bus 137. Bus 137 carries the data to main memory 134, from which processor 138 retrieves and executes the instructions. The instructions received by main memory 134 may optionally be stored on storage device 135 either before or after execution by processor 138.

Computer system 140 also includes a communication interface 141 coupled to bus 137. Communication interface 141 provides a two-way data communication coupling to a network link 139 that is connected to a local network 111. For example, communication interface 141 may be an Integrated Services Digital Network (ISDN) card or a modem to provide a data communication connection to a corresponding type of telephone line. As another non-limiting example, communication interface 141 may be a local area network (LAN) card to provide a data communication connection to a compatible LAN. For example, Ethernet based connection based on IEEE802.3 standard may be used such as 10/100 BaseT, 1000 BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (June 1999), “Internetworking Technologies Handbook”, Chapter 7: “Ethernet Technologies”, pages 7-1 to 7-38, which is incorporated in its entirety for all purposes as if fully set forth herein. In such a case, the communication interface 141 typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (Feb. 20, 2004), which is incorporated in its entirety for all purposes as if fully set forth herein.

Wireless links may also be implemented. In any such implementation, communication interface 141 sends and receives electrical, electromagnetic or optical signals that carry digital data streams representing various types of information.

Network link 139 typically provides data communication through one or more networks to other data devices. For example, network link 139 may provide a connection through local network 111 to a host computer or to data equipment operated by an Internet Service Provider (ISP) 142. ISP 142 in turn provides data communication services through the world wide packet data communication network Internet 11. Local network 111 and Internet 11 both use electrical, electromagnetic or optical signals that carry digital data streams. Also, satellite and network satellite communication and modules may be implemented. The signals through the various networks and the signals on the network link 139 and through the communication interface 141, which carry the digital data to and from computer system 140, are exemplary forms of carrier waves transporting the information.

A received code may be executed by processor 138 as it is received, and/or stored in storage device 135, or other non-volatile storage for later execution. In this manner, computer system 140 may obtain application code in the form of a carrier wave.

The concept of rapid classification of DNA for applications in various clinical, agricultural, environmental and military/forensic scenarios are disclosed herein, and may be implemented and utilized with the related processors, networks, computer systems, internet, and components and functions according to the schemes disclosed herein.

The methods and computer implemented methods of the disclosure can be implemented on the computers, systems and architecture described herein. The methods may be implemented by a computer program or programs stored on a computer readable medium such that the program causes the computer to carry out the methods and steps set forth above.

Although some embodiments have been described, the same and other embodiments are described in the Examples below, which are meant to illustrate, but not limit the invention.

### EXAMPLES

Example 1: Primers for rapid classification of E. coli isolates into Clermont phylogroups. 15,278 E. coli genomes were split between a training set of 14, 638 public genomes from BV-BRC and RefSeq using quality control measures as described in Hyun et al. (BMC Genomics 23(1):7 (2022)) and a validation set of 640 internal genomes. All genomes were assigned phylogroup annotations in line with current practice using ClermonTyping. 293, 826 genes were identified through pangenome construction and were filtered for those potentially associated with the phylogroup classification (defined as: there exists a pair of classes for which the gene is observed in X % of class 1 genomes and Y % of class 2 genomes, where X>Y+10), yielding 14,836 genes.

Genetic algorithms (GAs) were applied to iteratively identify genes that (1) both accurately discriminate phylogroups and (2) can be reliably identified by a marker sequence, given all observed variants and against the background of the E. coli pangenome. In a single iteration of this process, GAs were first trained to identify 4-8 genes from this set that best reproduces phylogroup classification, randomly seeded with seed=1-10 for a total of 50 GA models. Performance was quantified by treating all possible presence/absence combinations of a given gene set as a predicted cluster and computing the purity of the resulting cluster predictions against true phylogroups for all genomes. Fitness was defined as

$$\frac{1}{( 1 - purity )}$$

to enable smaller improvements closer to 100% purity to continue translating to larger fitness gains. GAs were implemented using PyGAD with the following parameters (num_generations=200, sol_per_pop=512, num_parents_mating=256, keep_elitism=1, crossover_type=‘uniform’, mutation_type=‘random’, mutation_probability=0.20, parent_selection_type=‘sss’), trained on the public genomes and evaluated on the internal genomes. Across all trained GAs, solutions at generation 110, 120, . . . , 200 were extracted and genes selected in at least three solutions were identified as candidates.

For each candidate gene, all 25 nt subsequences or “kmers” were counted for all observed variants of the gene, and kmers occurring in at least 90% of variants weighted by frequency were identified as primer candidates. 100 genomes were randomly sampled for each gene with balanced representation of genomes with/without the gene and across all phylogroups, and the presence/absence of primer candidates in the sampled genomes was used to compute the F1 score of each primer candidate at recovering its corresponding gene. Genes for which no primer candidate with F1>90% were removed, completing an iteration of the marker identification process.

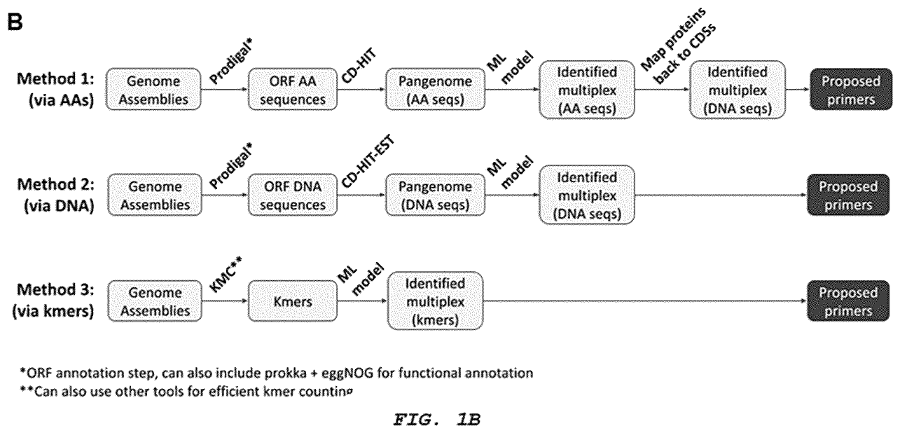

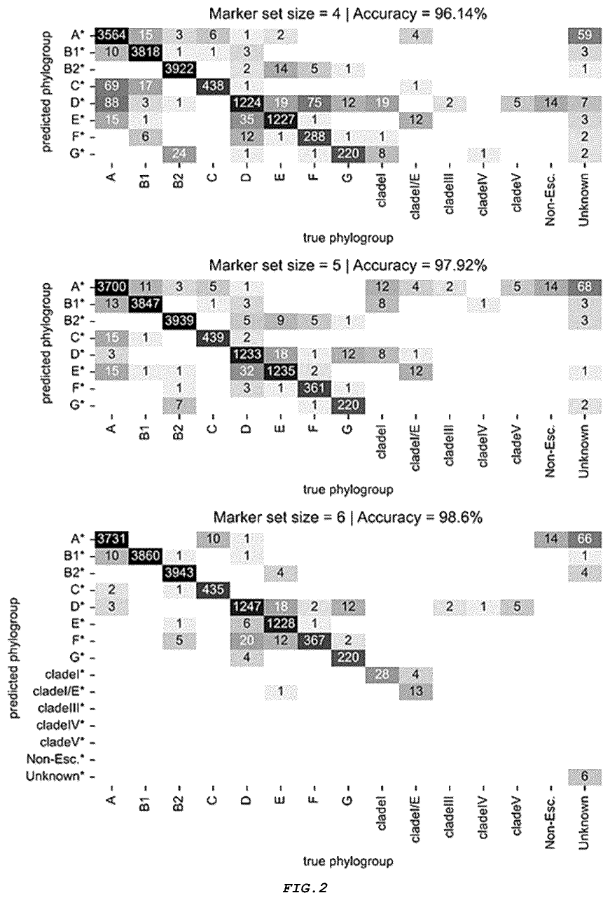

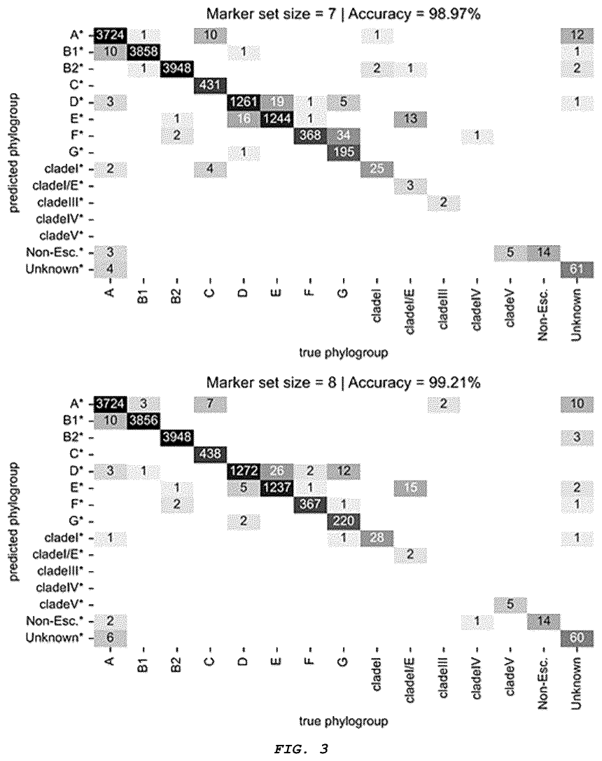

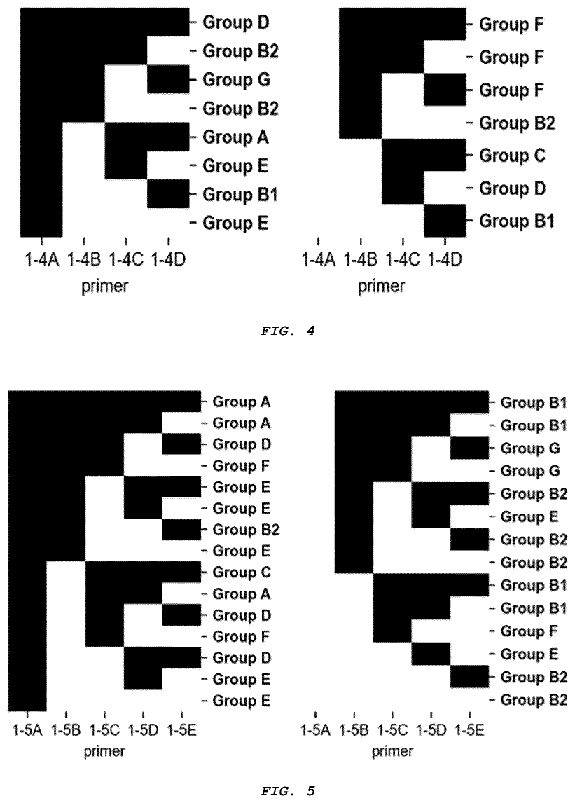

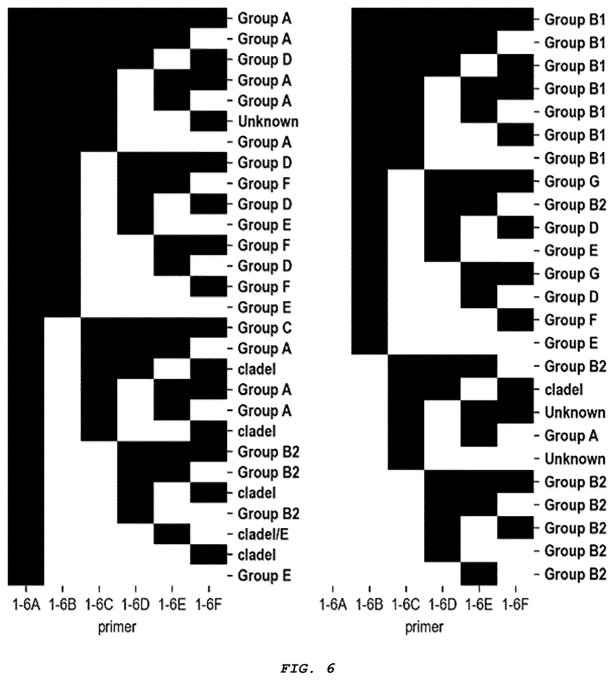

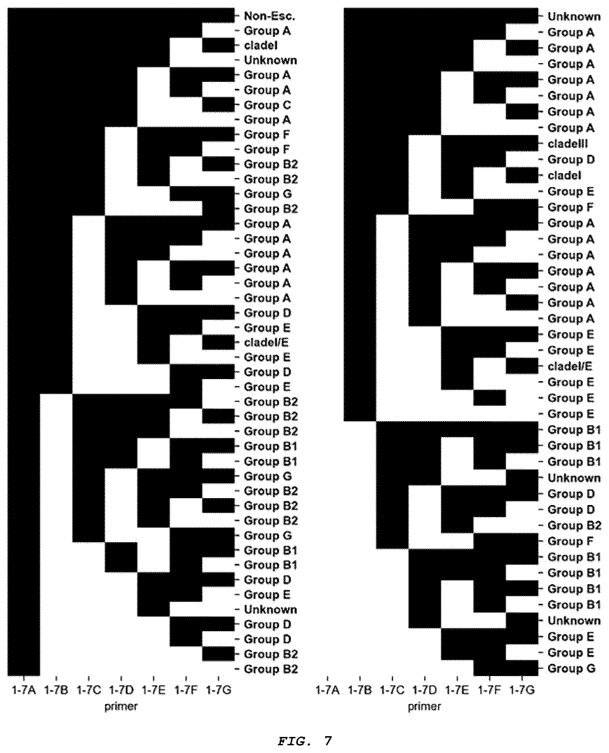

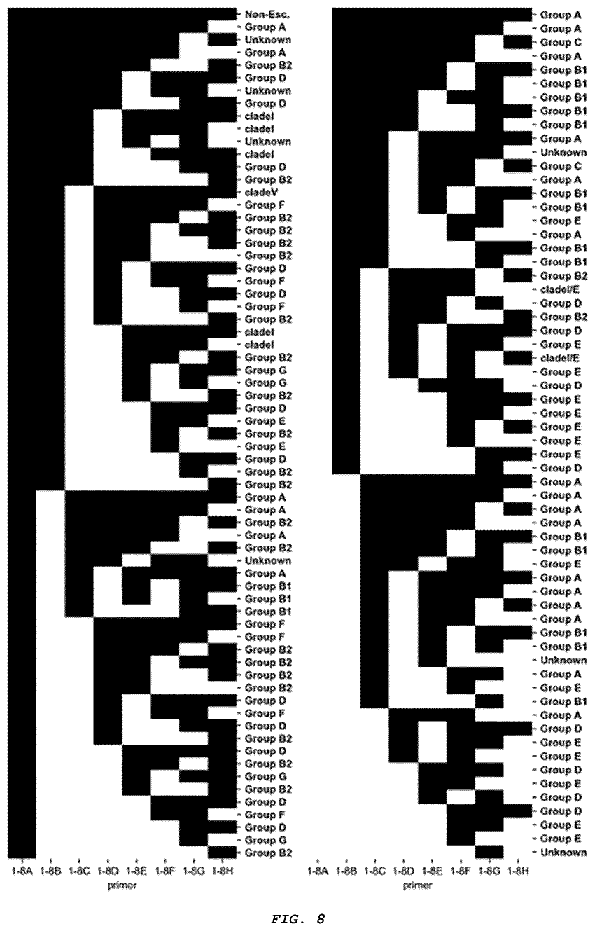

A total of six iterations were completed, after which all primer candidates across all iterations with F1>80% were identified. The presence/absence of these primer candidates across all genomes was computed, and GAs were similarly trained to identify 4-8 primers from this set that best discriminate phylogroups (using identical parameters as before, except num_generations=1000). The primers selected by the most accurate GA for each primer set size (4-8) are included as the final set of marker sequences for classifying E. coli by phylogroup. The marker sequences for each size (4-8) are available in Table 1, with annotations for corresponding genes derived from eggNOG-mapper. Confusion matrices describing the performance of each marker set presented in FIG. 2 and FIG. 3. Interpretation of marker sequences to predict E. coli phylogroup are described in FIGS. 4-8.

TABLE 1
Sets of marker sequences for the identification of
E. coli phylogroups. For each marker, the predicted protein product
of the targeted gene, gene name, and gene identifiers are shown
when available. Markers 4B and 8A (starred) are identical.

| ID | Primer | Primer Target Description | Gene | bnum | COG | KEGG |
|---|---|---|---|---|---|---|
| 1-4A | TGCAATACCGCATTCATCACCTCCG | Regulator of protease activity, | hflC | — | COG0330 | — |
| | (SEQ ID NO: 1) | stomatin/prohibitin superfamily | | | | |
| 1-4B* | TTATGGCGGCGATTATCGTCATGGC | Uncharacterized small protein | yldA | b4734 | — | — |
| | (SEQ ID NO: 2) | | | | | |
| 1-4C | GTGCTTGCAGCACATGTTGCGGATC | DNA-binding transcriptional | yiaG | b3555 | COG2944 | K07726 |
| | (SEQ ID NO: 3) | regulator | | | | |
| 1-4D | TTTCCTGTTATTCGGTAATGGCGAT | Predicted peptidase | abgB | — | COG1473 | K01451 |
| | (SEQ ID NO: 4) | | | | | |
| 1-5A | GTTGTCACAATCTAAATTTGCCGAT | DNA-binding transcriptional | yiaG | b3555 | COG2944 | K07726 |
| | (SEQ ID NO: 5) | regulator | | | | |
| 1-5B | CAGTTAACGAAGCCGATTGATCATT | Regulator of protease activity, | hflC | — | COG0330 | — |
| | (SEQ ID NO: 6) | stomatin/prohibitin superfamily | | | | |
| 1-5C | GGCGGTTTCCTGTTATTCGGTAATG | Predicted peptidase | abgB | — | COG1473 | K01451 |
| | (SEQ ID NO: 7) | | | | | |
| 1-5D | CGCGATATCGGTCTCGACGGCGTCG | Coproporphyrinogen III | hutW | — | COG0635 | K21936 |
| | (SEQ ID NO: 8) | oxidase | | | | |
| 1-5E | GCCGATGATTTATTTGATAAATTAG | Antitoxin component of type II | dinJ | b0226 | COG3077 | K07473 |
| | (SEQ ID NO: 9) | toxin-antitoxin system (YafQ- | | | | |
| | | DinJ) | | | | |
| 1-6A | ATGCGGTGATACAACGCTTCACCTT | Acetyl esterase/lipase, | aes | b0476 | COG0657 | K01066 |
| | (SEQ ID NO: 10) | alpha/beta hydrolase fold | | | | |
| 1-6B | GAATGAGGGCGGTTTGTCACAGGTT | Stress response protein, | yjaA | b4011 | 2DQT3 | — |
| | (SEQ ID NO: 11) | single-species biofilm | | | | |
| | | formation | | | | |
| 1-6C | TATCCCAGACAGTATCGAATTTATC | Heme iron utilization protein | hutX | — | COG3721 | K07227 |
| | (SEQ ID NO: 12) | | | | | |
| 1-6D | TACTGGCCGGGATGGGGCTGACCAT | Antitoxin component of type II | dinJ | b0226 | COG3077 | K07473 |
| | (SEQ ID NO: 13) | toxin-antitoxin system (YafQ- | | | | |
| | | DinJ) | | | | |
| 1-6E | GCCTACCAGTAATGGCTTGATATGG | Methylaspartate ammonia- | mal | — | COG3799 | K04835 |
| | (SEQ ID NO: 14) | lyase | | | | |
| 1-6F | GCGATTGTGGGTGACGCGGTCAATT | SNARE-associated | dedA | b2317 | COG0586 | K03975 |
| | (SEQ ID NO: 15) | membrane protein | | | | |
| 1-7A | TACCGATAAACTTTTACGATTTGAG | Toxic component of type II | yafQ | b0225 | COG3041 | K19157 |
| | (SEQ ID NO: 16) | toxin-antitoxin system (YafQ- | | | | |
| | | DinJ) | | | | |
| 1-7B | GACATTACTCCCGACGATAATTTAT | Acetyl esterase/lipase, | aes | b0476 | COG0657 | K01066 |
| | (SEQ ID NO: 17) | alpha/beta hydrolase fold | | | | |
| 1-7C | TCCAGCCATGTTTTATACCATGTTA | Ankyrin repeat protein | arpA | b4017 | COG0666 | K06867 |
| | (SEQ ID NO: 18) | | | | | |
| 1-7D | TACCGTCTGCGAATGGGGCAACGTC | Heme utilization protein | hutX | — | COG3721 | K07227 |
| | (SEQ ID NO: 19) | ChuX/HutX | | | | |
| 1-7E | GTCATTTATATCAACTCATCACGCG | TRAP-type C4-dicarboxylate | yiaM | b3577 | COG3090 | K21394 |
| | (SEQ ID NO: 20) | transporter subunit | | | | |
| 1-7F | CCAATTTCTTTGTTGCAGAAAAAGT | Stress response protein, | yjaA | b4011 | 2DQT3 | — |
| | (SEQ ID NO: 21) | single-species biofilm | | | | |
| | | formation | | | | |
| 1-7G | CGCGATTGTGGGTGACGCGGTCAAT | SNARE-associated | dedA | b2317 | COG0586 | K03975 |
| | (SEQ ID NO: 22) | membrane protein | | | | |
| 1-8A* | TTATGGCGGCGATTATCGTCATGGC | Uncharacterized protein | yldA | b4734 | — | — |
| | (SEQ ID NO: 23) | | | | | |
| 1-8B | TAAGAATGAAGAGAAATCACTAAAC | Uncharacterized protein | yrhA | — | 292QA | — |
| | (SEQ ID NO: 24) | | | | | |
| 1-8C | ATGGTTATTTCTTGATGAACCAACT | ABC-type hemin transporter | hmuV | — | COG4559 | K02013 |
| | (SEQ ID NO: 25) | subunit (hmuTUV) | | | | |
| 1-8D | CACGGTGCTTGTCGATCTGAACGAT | PfkB family carbohydrate | scrK | — | COG0524 | K00847 |
| | (SEQ ID NO: 26) | kinase | | | | |
| 1-8E | GCAGCCGTCGCGGGGATGTCTGGTG | Glutamate mutase epsilon | glmE | — | COG4865 | K19268 |
| | (SEQ ID NO: 27) | subunit | | | | |
| 1-8F | GAATGCCCCGACATTACTCCCGACG | Acetyl esterase/lipase, | aes | b0476 | COG0657 | K01066 |
| | (SEQ ID NO: 28) | alpha/beta hydrolase fold | | | | |
| 1-8G | GCATGGATGTGGAACTGGCGATATT | Stress response protein, | yjaA | b4011 | 2DQT3 | |
| | (SEQ ID NO: 29) | single-species biofilm | | | | |
| | | formation | | | | |
| 1-8H | TTTATCGGTAAGTTTGTAAATCAGG | Toxic component of type II | yafQ | b0225 | COG3041 | K19157 |
| | (SEQ ID NO: 30) | toxin-antitoxin system (YafQ- | | | | |
| | | DinJ) | | | | |

Example 2: Primers for rapid classification of S. aureus isolates into clonal complexes. S. aureus genomes of high quality and with clonal complex metadata were identified from the BV-BRC database, resulting in 753 genomes. The genomes spanned 14 clonal complexes with at least 7 genomes each, as well as 8 other rarer clonal complexes which were grouped together as “other”. 10% of genomes were randomly selected to be the validation set, with the remaining 90% as the training set. A S. aureus pangenome was prepared similarly to the previous example, and genes were filtered for those present in at least three genomes or missing in at least three genomes, resulting in 6,990 genes.

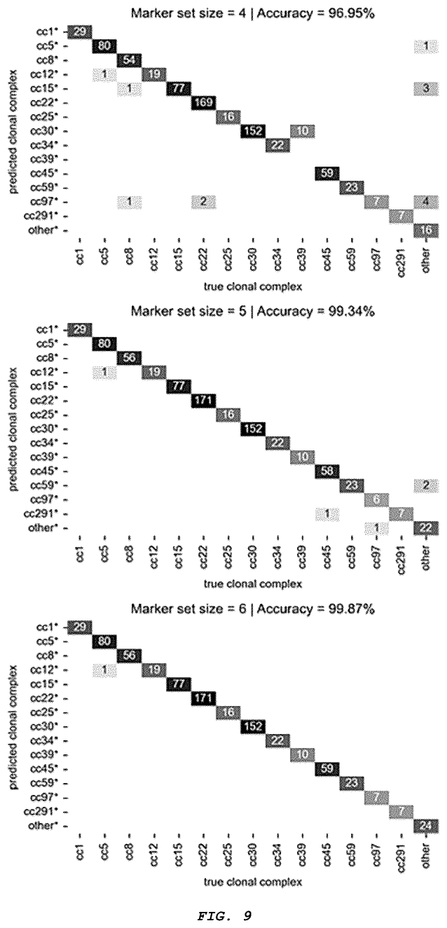

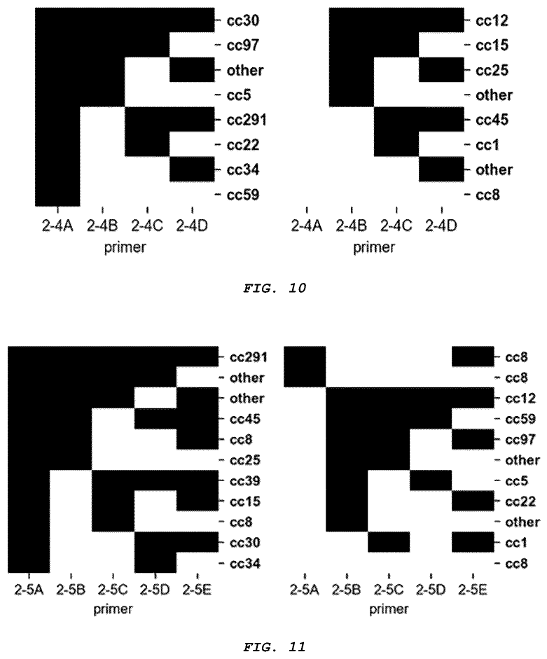

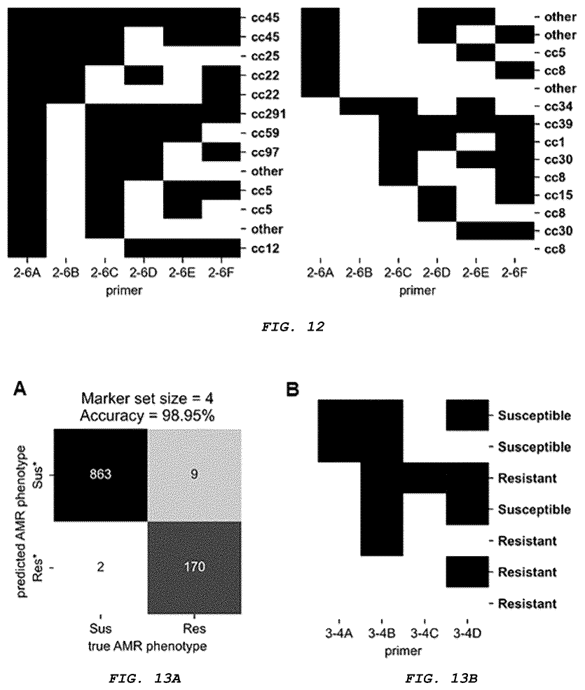

Sets of genes and marker sequences were iteratively identified using the same genetic algorithm approach as the previous example and as described in FIG. 1, with a total of four iterations completed. The marker sequences were developed for sets of size 4, 5, and 6, and are presented in Table 2. Annotations for corresponding genes are supplemented with results from blastp results from NCBI, due to poor coverage from eggNOG-mapper. Confusion matrices describing the performance of each marker set are presented in FIG. 9. Interpretation of marker sequences to predict S. aureus clonal complex are described in FIGS. 10-12.

TABLE 2
Sets of marker sequences for the identification of S. aureus
clonal complexes. For each marker, the predicted protein product of
the targeted gene and gene identifiers are shown when available.
“RefSeq/GenBank” refers to the most common variant of the gene
targeted by the primer.

| ID | Primer | Primer Target Description | RefSeq/GenBank | COG |
|---|---|---|---|---|
| 2-4A | CATGACATGTTCGTAAATGATGATT | Staphylococcal enterotoxin type 26 | WP_001622271.1 | 2CC33 |
| | (SEQ ID NO: 31) | | | |
| 2-4B | TCTGAAAAACCAAATTGTACAGACG | MFS transporter | WP_000130758.1 | COG0477 |
| | (SEQ ID NO: 32) | | | |
| 2-4C | CAAATGTATAAATAATAAATGCTAT | Uncharacterized membrane protein | WP_000956429.1 | COG4858 |
| | (SEQ ID NO: 33) | | | |
| 2-4D | TTAGATAATTATTTAGTATTAGCAT | HTH-type transcriptional regulator | ADC38645.1 | COG1846 |
| | (SEQ ID NO: 34) | SarT | | |
| 2-5A | TTAGAATCTTTTGCCTTTACCGCAT | LPXTG-anchored surface protein | AYV00666.1 | — |
| | (SEQ ID NO: 35) | SasK | | |
| 2-5B | TACAAAATTAAAATTAAATGGATAT | Hypothetical protein | WP_000410720.1 | — |
| | (SEQ ID NO: 36) | | | |
| 2-5C | CTTAAGCATAAAAATTTATATGAAT | Staphylococcal enterotoxin type U | WP_000764690.1 | 2CC33 |
| | (SEQ ID NO: 37) | | | |
| 2-5D | TCAATTTTATAGTCTGTAGTCTTTG | Iron-hydroxamate ABC transporter | WP_000825510.1 | COG0614 |
| | (SEQ ID NO: 38) | substrate-binding protein | | |
| 2-5E | TGATCAGCCATTGACTTAATCGGTG | Uncharacterized membrane protein | WP_000956429.1 | COG4858 |
| | (SEQ ID NO: 39) | | | |
| 2-6A | CATTTAACTGATTAGTATCTAATTT | Hypothetical protein | WP_000410720.1 | — |
| | (SEQ ID NO: 40) | | | |
| 2-6B | ATTGGGACAATATTATTAAAAGCAT | ECF-type riboflavin transporter | WP_000743714.1 | COG4720 |
| | (SEQ ID NO: 41) | substrate-binding protein | | |
| 2-6C | CATTAAAAAAGATTCATAAAGGAAT | RES family NAD+ phosphorylase | WP_103145692.1 | — |
| | (SEQ ID NO: 42) | | | |
| 2-6D | TTCAATTGTTCTGGTTTAGGATTGC | Staphylococcal enterotoxin type U | WP_000764690.1 | 2CC33 |
| | (SEQ ID NO: 43) | | | |
| 2-6E | TATAGTCTGTAGTCTTTGTCGAGTT | Iron-hydroxamate ABC transporter | WP_000825510.1 | COG0614 |
| | (SEQ ID NO: 44) | substrate-binding protein | | |
| 2-6F | AATAACAATTCAACACGTAATTTTT | Uncharacterized membrane protein | WP_000956429.1 | COG4858 |
| | (SEQ ID NO: 45) | | | |

Example 3: Primers for determining resistance against ciprofloxacin for E. coli isolates in the B2 phylogroup. E. coli genomes from Example 1 were filtered to those with antimicrobial resistance metadata against ciprofloxacin available on BV-BRC, resulting in 1044 genomes (179 resistant, 865 susceptible). To capture mechanisms of resistance conferred by point mutations, the initial feature set was expanded from genes to also include individual amino acid variants of those genes or “alleles”, resulting in 267, 328 initial genetic features. These features were pre-filtered based on association with the resistance phenotype similarly to Example 1, yielding 13,953 features that satisfy: the feature is present in X % of resistant genomes and Y % of susceptible genomes and |X−Y|>10.

Smaller sets of features predictive of resistance were identified using the same genetic algorithm approach as the previous examples. GA models from 10 different randomization states were trained to identify 4-8 features that distinguish resistant from susceptible genomes, for a total of 50 models. Across all trained GAs, solutions at generation 110, 120, . . . , 200 were extracted and features selected in at least three solutions were identified as candidate features to target. Primer candidates for each feature were identified using the same kmer counting and F1 score filtering approach as the previous examples. Only a single iteration of the full workflow described in FIG. 1 was completed before sufficiently accurate primer sets were identified. However, it is expected that improvement in the accuracy of the primer set can be realized with additional iterations and/or longer GA generation limits like in Examples 1-2. A single set of marker sequences (size 4) is available in Table 3 with annotations for corresponding genes derived from eggNOG-mapper and NCBI blastp. Larger primer sets were excluded as they targeted similar sets of genes and did not confer meaningful improvements to accuracy. The confusion matrix and interpretation of this marker sequence set is available in FIG. 13. Notably, the GA approach independently selected primers targeting regions of gyrA and parC, which are known determinants of ciprofloxacin resistance.

TABLE 3
Four marker sequences for the determination of resistance
against ciprofloxacin in E. coli B2 strains. For each primer, the
predicted protein product of the targeted gene, gene name, and gene
identifiers are shown when available. The target of primer 3-4D is an
undercharacterized protein represented by ESA89826.1 (GenBank) .

| ID | Primer | Primer Target Description | Gene | bnum | COG | KEGG |
|---|---|---|---|---|---|---|
| 3-4A | TTCGTGGTATTCGTTTAGGCGAAGG | DNA gyrase subunit A | gyrA | b2231 | COG0188 | K02469 |
| | (SEQ ID NO: 46) | | | | | |
| 3-4B | TAACAGGCAATATCGCCGTGCGGAT | DNA topoisomerase IV subunit | parC | b3019 | COG0188 | K02621 |
| | (SEQ ID NO: 47) | A | | | | |
| 3-4C | GTTATCGCGATGAATATAAACTGGC | Putative FAD-linked | ydiJ | b1687 | COG0247 | — |
| | (SEQ ID NO: 48) | oxidoreductase | | | | |
| 3-4D | CAGCATGGCCCATCCTACTGAAACT | Abortive infection family protein | — | — | — | — |
| | (SEQ ID NO: 49) | | | | | |

### Validation of Example 3: Marker Sequences for Determining Resistance Against Ciprofloxacin for E. coli Isolates in the B2 Phylogroup

Selection of a genetically and phenotypically diverse set of strains for marker validation. A set of 442 B2 E. coli strains not used for marker sequence design were characterized as follows. Starting with the strains' genome assemblies, pairwise genetic distances were computed by using MASH (Ondov et al., Genome Biol, 17:132, 2016), which were then used to generate a hierarchical clustering (average linkage) of the strains and assign the strains to one of 23 resulting genetic clusters. AMR phenotypes (binary ciprofloxacin resistant/susceptible calls) were predicted in silico and independently of the marker sequences by first constructing a pangenome combining 1260 publicly available B2 strains with ciprofloxacin resistance data from BV-BRC (Olson et al., Nucl. Acids. Res., 51: D678-689, 2023) with the 442 candidate validation strains. An XGBoost machine learning model was trained on the 1260 BV-BRC strains to predict ciprofloxacin resistance from pangenomic features (n_estimators=32, max_depth=3, colsample_bytree=0.75, subsample=0.75), achieving mean test MCC=0.95 and accuracy=0.98 during 5-fold cross validation. This model was then used to assign predicted AMR phenotypes to the 442 candidate validation strains.

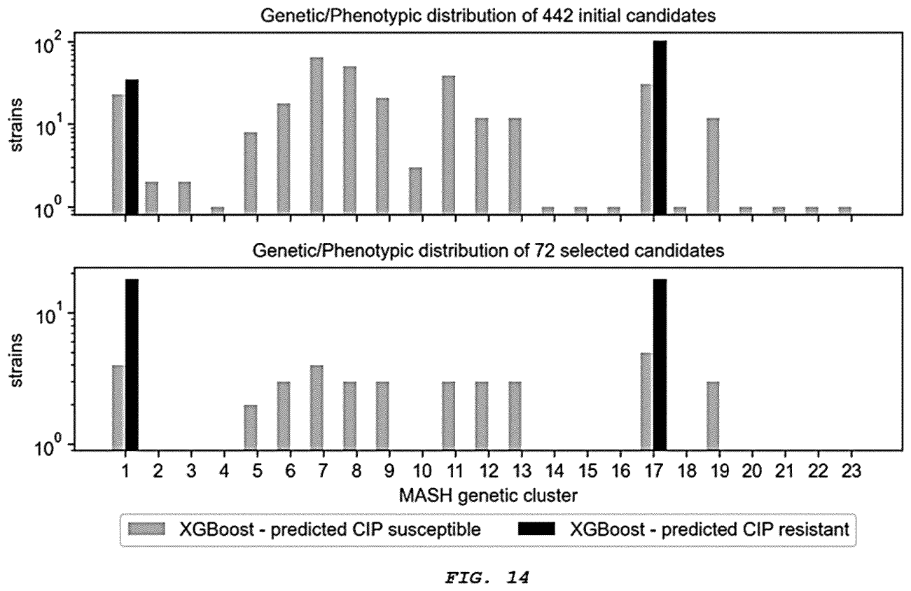

From the characterized 442 candidate validation strains, a balanced set of 72 strains was selected by randomly selecting 2-4 predicted-susceptible strains from each MASH cluster to a total of 36 strains, and similarly for 36 predicted-resistant strains. Clusters with fewer than four strains were excluded. Only two MASH clusters had any predicted-resistant strains, compared to 11 with predicted-susceptible strains (FIG. 14).

Characterization of proposed marker sequences against 72 validation strains. The four marker sequences were aligned to the genome assemblies of the 72 validation strains and interpreted against their corresponding consensus sequences.
3-4A: Targets a stable region of gyrA (DNA gyrase subunit A) matching the consensus sequence. SNPs against the consensus are observed rarely in 3/25 positions covered by this sequence.
3-4B: Targets part of the quinolone resistance-determining region of parC (DNA topoisomerase IV subunit A), capturing a single SNP against the consensus that yields the resistance mutation S80I (AGT->ATT).
3-4C: Targets a stable region of ydiJ (putative FAD-linked oxidoreductase). Aligned exactly in all 72 validation strains. Was previously found missing very rarely in the initial training strains used to develop the marker sequences.
3-4D: Targets a stable region of rarely detected abi (abortive infection family protein). Aligned in only 6 validation strains, and all such alignments were exact matches.

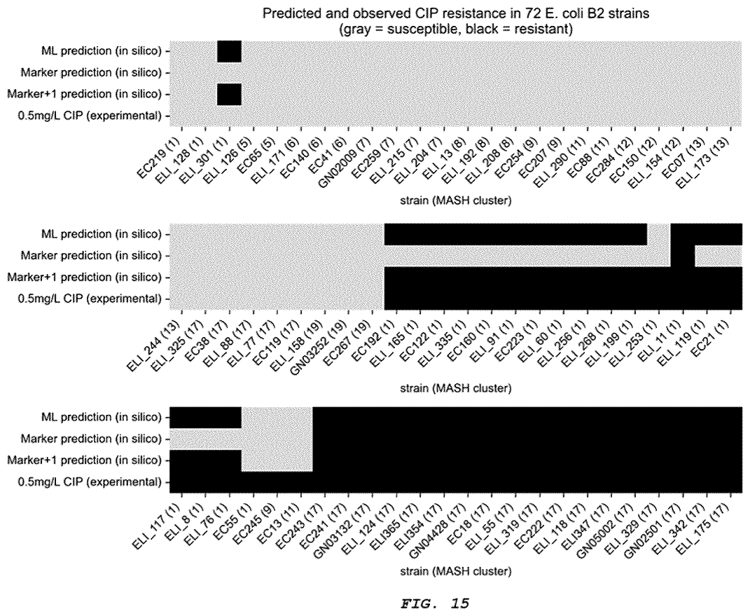

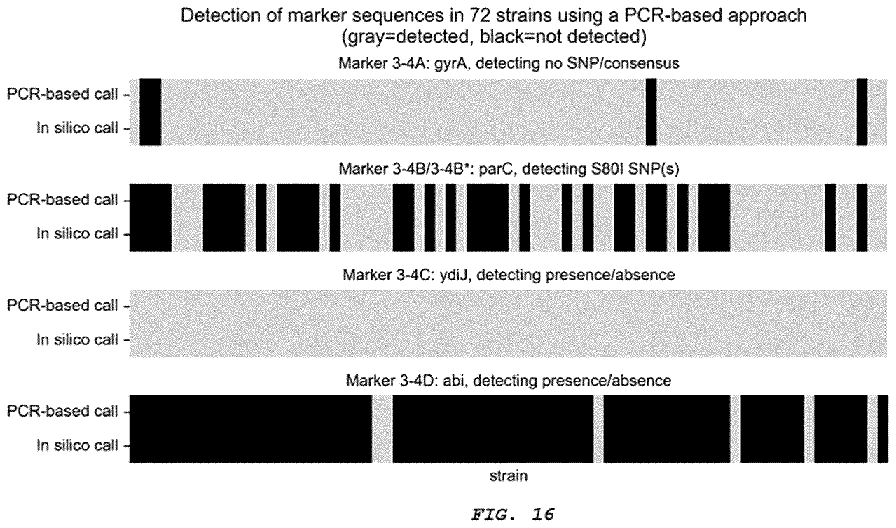

Ciprofloxacin resistance testing of validation strains. Ciprofloxacin resistance was measured experimentally in growth/no growth experiments with 0.5 mg/L CIP. Each strain isolate was grown on CA-MHB media at 37° C., transferred to CA-MHB+ciprofloxacin media to an initial density of OD=0.10 and ciprofloxacin concentration of 0.5 mg/L, then incubated at 37° C. for 48 hours with shaking at 800 rpm and density measurements every 15 minutes (BioTek plate reader). 39 strains were resistant and 33 strains were susceptible, confirming the validation set to be nearly phenotypically balanced (FIG. 15).

Performance of the original proposed and modified marker sequences on validation strains. Ciprofloxacin resistance predictions based on the four original marker sequences were 72% accurate with 20 false negatives and 0 false positives (FIG. 15). Analysis of the errors found that 17/20 false negatives could be attributed to missing a double SNP mutation (AGT->ATC) that would yield the same S80I substitution as the single SNP captured by marker 3-4B. This double SNP is able to be captured by the addition of a 5th marker 3-4B* (TAACAGGCGATATCGCCGTGCGGAT (SEQ ID NO: 50)) with one base pair difference from marker 3-4B. Updating the interpretation of the four markers to accept both 3-4B or 3-4B* as being positive for 3-4B increases accuracy to 94% with 3 false negatives and 1 false positive (FIG. 15).

Development of a PCR assay for direct capture of marker sequences targets. A PCR-based approach was developed to capture the targets of the modified marker sequences without requiring whole genome sequencing, as follows. Templates were prepared by first making cleared lysates from plated cells. Each strain isolate was plated on LB and grown overnight. An amount roughly the size of a grain of sand was transferred to a 200 μL tube with 40 μL water, heated to 95° C. in a thermocycler with a heated lid for 5 minutes to lyse, and then centrifuged to pellet the cellular debris. Six 12.5 μL PCR reactions per strain were run using an equally divided master mix which included 1×SYBR dye and 3 μL of the cleared lysates but no primers. Six primer pairs (Table 4) were then added to the samples, one primer pair per well. For targets involving gyrA (marker 3-4A) and parc (markers 3-4B, 3-4B*) the Amplification Refractory Mutation System PCR (ARMS-PCR) (Little, Curr. Protoc. Hum. Genet., Ch. 9, Unit 9.8, 2001) was used to design primers that distinguish between the relevant variants. Both targets required two sets of primers, one to detect the dominant form of the region targeted by the marker sequence and the other to detect the variant described by the marker sequence. For targets involving ydiJ (marker 3-4C) and abi (marker 3-4D), standard PCR primers targeting the genes were used after verifying that they did not yield non-specific amplicons on strains lacking these genes.

TABLE 4
PCR primer sequences for detection of ciprofloxacin resistance marker sequences.

| Primer Pair | Related Marker | Primer Sequence |
|---|---|---|
| gyrA_mut_F | 3-4A (gyrA) | TTCTGTCCGTGCGATGGGCTGCAACACCACCGGTGTTCTC (SEQ ID |
| gyrA_R | | NO: 51) |
| | | ACGCGACTTGGTTGGGTATTC (SEQ ID NO: 52) |
| gyrA_WT_F | 3-4A (gyrA) | TTCTGTCCGTGCGATGGGCTGCAACACCACCGGTGTTCTT (SEQ ID |
| gyrA_R | | NO: 53) |
| | | ACGCGACTTGGTTGGGTATTC (SEQ ID NO: 54) |
| parC_F | 3-4B/3-4B* (parC) | CGGGATTCGGTGTAACGCAT (SEQ ID NO: 55) |
| parC_mut_R | | GTACTGGGTAAATACCATCCGCACGGCGATCT (SEQ ID NO: 56) |
| parC_F | 3-4B/3-4B* (parC) | CGGGATTCGGTGTAACGCAT (SEQ ID NO: 57) |
| parC_WT_R | | GTACTGGGTAAATACCATCCGCACGGCGATCG (SEQ ID NO: 58) |
| ydiJ_F | 3-4C (ydiJ) | GACGCTGGAACAGCTTGAAGCTCT (SEQ ID NO: 59) |
| ydiJ_R | | GCCCAGAGCCAGTTTATATTCATCGCGATA (SEQ ID NO: 60) |
| abi F | 3-4D (abi) | CAGCATGGCCCATCCTACTG (SEQ ID NO: 61) |
| abi_R | | TGCCGCCATCCTGATCC (SEQ ID NO: 62) |

Promega GoTaq® G2 Colorless Master Mix, which lacks 3′ to 5′ proofreading activity, was used for all of the PCR reactions. Good performance was observed for both the hot start and standard versions of the master. When the standard version was used, the plate was loaded onto a block that was pre-heated to the initial denaturation temperature. The six primer pairs were initially run on their respective positive control templates using an annealing gradient of eight different temperatures. An annealing temperature was selected which optimized the likelihood of the gyrA and parc ARMS primer pairs working well on their intended targets but poorly or not at all on the non-targeted counterparts. The ydiJ and abi primers were adjusted to function well at the same temperature so that all six assays could be run on the same plate at the same time. The PCR reactions were all run in a 96-well format on a CFX Duet Real-Time PCR System. Thirty-five cycles were run in total, each cycle consisting of 10 seconds at 95° C., 15 seconds at 69° C., and 30 seconds at 72° C. Outcomes were scored by first setting a threshold to cross all of the amplification curves in their log-linear region, and calls were based on Cq numbers as follows: For gyrA and parC targets, the marker or variant was scored as positive if its Cq number was lower than its counterpart by at least 5 cycles. For samples whose amplification curves did not reach threshold, 35, the total number of cycles run, was used for the calculations. For ydiJ and abi targets, Cq<25 was considered positive.

Consistency between PCR assay and in silico marker sequence presence/absence calls derived from assembled genomes on validation strains. Against the 72 validation strains, the PCR-based approach was able to exactly match in silico presence/absence calls for each marker sequence based on assembled genomes (FIG. 16), enabling prediction of ciprofloxacin resistance with 94% accuracy without requiring whole genome sequencing.

A number of embodiments have been described herein. Nevertheless, it will be understood that various modifications may be made without departing from the spirit and scope of this disclosure. Accordingly, other embodiments are within the scope of the following claims.

1. A method to identify a set of oligonucleotide marker sequences for rapid classification of microbial pathogens from a dataset of microbial genome assemblies, comprising carrying out steps (1)-(8):
(1) clustering open reading frames by protein sequence to form a pangenome from the dataset of microbial genome assemblies, wherein individual clusters of open reading frames are designated as “genes”;
(2) filtering the genes to identify sets of genes that are associated by a targeted classification scheme;
(3) training machine learning models to iteratively identify minimal sets of related “genes” that are (a) able to accurately reproduce a targeted classification scheme, and (b) can be accurately identified by a marker sequence, given all observed variants of the gene and against the background of the pangenome, wherein the identified minimal sets of associated genes that meet criteria (a) and (b) are designated as candidate genes;
(4) identifying sequence variants from each of the candidate genes, and from which, subsequences having a defined number of nucleotides are enumerated, wherein the enumerated subsequences of length k are designated “kmers”;
(5) identifying conserved kmers for all candidate genes across the pangenome;
(6) filtering the conserved kmers to identify those conserved kmers that accurately reproduce the presence/absence of their corresponding gene, wherein these identified conserved kmers are designated as accurate kmers;
(7) training machine learning models to classify genomes from the accurate kmers and filtering out genes that cannot be reproduced accurately from a single accurate kmers, wherein steps (3)-(6) are repeated until a kmers-based machine learning model able to reproduce the target classification scheme with accuracy greater than 95%, 98%, or 99% is generated; and
(8) selecting a minimal set of oligonucleotide marker sequences from the accurate machine learning model, wherein the minimal set of oligonucleotide marker sequences can be used to generate primers for the classification of microbial pathogens.

2. The method of claim 1, wherein the microbial pathogens are selected from bacterial pathogens, viral pathogens, and fungi pathogens.

3. The method of claim 1, wherein the dataset comprises microbial genome assemblies from bacteria, viruses or fungi.

4. The method of claim 1, wherein for step (1), the microbial genomes assemblies contain genomes that have been filtered for quality based on genome status, size, number of contigs, number of genes and consistency.

5. The method of claim 1, wherein for step (1), the microbial genomes assemblies have been annotated to mark open reading frames in the genomes.

6. The method of claim 1, wherein for step (1), wherein the open reading frames are clustered using a program that clusters and compares protein or nucleotide sequences.

7. The method of claim 1, wherein for step (2), wherein the targeted classification scheme comprises genes that are related to drug-resistance, virulence, pathotype, or phylogenetically-defined subgroups.

8. The method of claim 1, wherein for step (2), wherein the genes are filtered for those associated with the classification scheme, wherein association is based upon a pair of classes for which the gene is observed in X % of class 1 genomes and Y % of class 2 genomes, where X>Y+10.

9. The method of claim 1, wherein for step (3), the machine learning models are trained to identify 4-8 genes that most accurately recover the targeted classification scheme.

10. The method of claim 1, wherein for step (4), the subsequences have a number of nucleotides selected from 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, and 35 nt, or a range of nucleotides that includes or is between any two of the foregoing numbers.

11. The method of claim 1, wherein for step (4), the subsequences have a number of nucleotides selected from 20 nt to 30 nt.

12. The method of claim 1, wherein for step (5), wherein conserved kmers occur in greater than 80%, 85%, 90%, 91%, 92%, 93%, 94% or 95% of instances of the gene.

13. The method of claim 1, wherein for step (6), wherein the accuracy of the conserved kmers to reproduce the presence/absence of their corresponding gene is determined based upon the F1 score.

14. The method of claim 1, wherein for step (7), the machine learning models are trained to identify 4-8 kmers that most accurately recover the targeted classification scheme.

15. The method of claim 1, wherein the method further comprises:
classifying a microbial pathogen using one or more oligonucleotide marker sequences selected in step (8).

16. The method of claim 1, wherein the method is implemented by a computer.

17. The method of claim 16, wherein the computer system is linked to an oligonucleotide synthesizer.

18. A computer readable medium comprising instructions to cause a computer to identify a set of oligonucleotide marker sequences for rapid classification of microbial pathogens from a dataset of microbial genome assemblies, the computer instructions causing the computer to:
(1) cluster open reading frames by protein sequence to form a pangenome from the dataset of microbial genome assemblies, wherein individual clusters of open reading frames are designated as “genes”;
(2) filter the genes to identify sets of genes that are associated by a targeted classification scheme;
(3) train machine learning model(s) to iteratively identify minimal sets of related “genes” that are (a) able to accurately reproduce a targeted classification scheme, and (b) can be accurately identified by a marker sequence, given all observed variants of the gene and against the background of the pangenome, wherein the identified minimal sets of associated genes that meet criteria (a) and (b) are designated as candidate genes;
(4) identify sequence variants from each of the candidate genes, and from which, subsequences having a defined number of nucleotides are enumerated, wherein the enumerated subsequences of length k are designated “kmers”;
(5) identify conserved kmers for all candidate genes across the pangenome;
(6) filter the conserved kmers to identify those conserved kmers that accurately reproduce the presence/absence of their corresponding gene, wherein these identified conserved kmers are designated as accurate kmers;
(7) train machine learning models to classify genomes from the accurate kmers and filtering out genes that cannot be reproduced accurately from a single accurate kmers, wherein steps (3)-(6) are repeated until a kmers-based machine learning model able to reproduce the target classification scheme with accuracy greater than 95%, 98%, or 99% is generated; and
(8) select a minimal set of oligonucleotide marker sequences from the accurate machine learning model, wherein the minimal set of oligonucleotide marker sequences can be used to generate primers for the classification of microbial pathogens.

19. A catalog of oligonucleotide marker sequences obtained by the method of claim 1.

20. A method of identifying a pathogen in a sample, the method comprising comparing sequence reads obtained from the genome of the pathogen in the sample to the catalog of oligonucleotide markers of claim 19 to identify the pathogen with a complementary sequence.
